(19) United States
(12) Patent Application Publication
(10) Pub. No.: US 2024/0090600 A1
(43) Pub. Date: Mar. 21, 2024

(54) WEARABLE ELECTRONIC GARMENTS AND METHODS OF MAKING SAME

(71) Applicant: Battelle Memorial Institute, Columbus, OH (US)

(72) Inventors: Matthew Colachis, Columbus, OH (US); Katherine M. Palmer, Galloway, OH (US); Nicholas Annetta, Columbus, OH (US); Samuel Colachis, Columbus, OH (US); Charli Ann Hooper, Pittsburgh, PA (US)

(21) Appl. No.: 18/368,785

(22) Filed: Sep. 15, 2023

Related U.S. Application Data
(60) Provisional application No. 63/407,216, filed on Sep. 16, 2022.

(51) Int. Cl. A41D 13/12; D03D 1/00; D04B 1/24

(52) U.S. Cl. CPC A41D 13/1281; A41D 13/1245; D03D 1/0088; D04B 1/24; A41D 2500/10; A41D 2500/20; D10B 2403/02431

(57) ABSTRACT

An electronic garment includes an elastic textile garment configured to be worn on anatomy of an associated wearer. The elastic textile garment has an inner surface arranged to contact the anatomy when the elastic textile garment is worn on the anatomy. Electrodes are secured to the inner surface of the elastic textile garment. Each electrode includes at least an exposed portion of an insulated electrically conductive thread that is sewn onto or into the elastic textile garment. An electrically conductive polymer electrode material, such as a mixed ionic-electronic conducting (MIEC) material, is arranged to contact the electrodes, for example as an inner compression sleeve liner coated or infused with the MIEC material. The elastic textile garment and the electrically conductive threads may be formed together by three-dimensional (3D) knitting. A scent may be added to the elastic textile garment.

This application claims priority to U.S. Provisional Patent Application Ser. No. 63/407,216 filed Sep. 16, 2022 and titled “WEARABLE ELECTRONIC GARMENTS AND METHODS OF MAKING SAME”, which is incorporated by reference in its entirety.

### BACKGROUND

The following relates to the wearable electronic garment arts, electrophysiology measurement arts such as electromyography (EMG) measurement arts, transcutaneous electrical nerve stimulation (TENS) arts including functional electrical stimulation arts, and to the like.

Clothing that incorporates electrodes have a variety of uses in sports rehabilitation, transcutaneous electrical nerve stimulation (TENS), functional electrical stimulation (FES), and so forth. In some designs of an electronic garment for such purposes and others, the electrodes are made of a metal such as stainless steel, and an electrolytic conductive gel disposed on the electrode bridges the ionic collector (skin) with the current collector to reduce the electrical potential drop between the metal electrode and the skin. If no conductive gel is used, or if the conductive gel is insufficient or gets wiped off (for example during donning of the garment), then the person wearing the electronic garment may experience pain or skin irritation, for example due to electrical arcing between the metal electrode and the skin. Conductive gels can also introduce other problems such as allergic reactions, discomfort, and variable signal quality that changes as a function of gel loading. The conductive gel can also be messy and difficult to wash away. The pecuniary cost of hydrogels, supply maintenance, setup time, and ease-of-use of these gels are additional problems.

Additional issues that can arise with electronic garments include: the time it takes to don and doff the garment, the ability of the garment to conform to the user's skin especially when mechanical stressors are acted upon them (i.e., movement/sport applications), breathability of the garment, washability of the garment, and the weight of the garment. It will be appreciated that poor conformance of the garment with the skin can contribute to poor electrical contact between the electrodes and the skin, again potentially leading to electrical arcing or similar issues. Some electronic garments also are unwieldy or consist of multiple parts.

Certain improvements are disclosed herein.

### BRIEF SUMMARY

In accordance with some illustrative embodiments disclosed herein, an electronic garment comprises: an elastic textile garment configured to be worn on anatomy of an associated wearer, the elastic textile garment comprising an elastic textile and having an inner surface arranged to contact the anatomy when the elastic textile garment is worn on the anatomy; electrodes secured to the inner surface the elastic textile garment, each electrode including an electrically exposed portion of an insulated electrically conductive thread sewn onto or into the elastic textile garment; and an electrically conductive polymer electrode material arranged to contact the electrodes. In some embodiments, the electrically conductive polymer electrode material comprises a mixed ionic-electronic conducting (MIEC) material. In some embodiments each electrode further includes a flexible electrically conductive layer sewn onto the inner surface of the elastic textile garment, the electrically exposed portion of the insulated electrically conductive thread being electrically connected with the flexible electrically conductive layer. In some such embodiments, the flexible metal layers of the electrodes are electrically conductive meshes.

In accordance with some illustrative embodiments a method of manufacturing an electronic garment is disclosed, the method comprising: providing an elastic textile garment configured to be worn on anatomy of an associated wearer, the elastic textile garment comprising an elastic textile and having an inner surface arranged to contact the anatomy when the elastic textile garment is worn on the anatomy; sewing electrodes comprising electrically exposed portions of electrically conductive threads onto the elastic textile garment; and arranging an electrically conductive polymer material to contact the electrodes. In some embodiments the electrically conductive polymer material comprises an MIEC material. In some embodiments the electrodes further include electrically conductive meshes, and the sewing of the electrodes onto or into the elastic textile garment includes: sewing the electrically conductive threads onto or into the elastic textile garment; and sewing the electrically conductive meshes onto the elastic textile garment; wherein the exposed portions of the electrically conductive threads are electrically connected with the electrically conductive meshes. In some embodiments the method further includes adding a scent to the elastic textile garment.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically illustrates an electronic garment comprising an armband.

FIG. 2 diagrammatically illustrates an electrical stimulation system and/or electrophysiology readout system including the armband of FIG. 1.

FIG. 3 diagrammatically illustrates an inner surface of a portion of an electronic garment according to one illustrative embodiment.

FIGS. 4 and 5 diagrammatically illustrate exploded perspective and perspective views, respectively, of an electrode of an electronic garment.

FIG. 6 diagrammatically illustrates an exploded perspective view of an electrode of an electronic garment according to another embodiment.

FIG. 7 diagrammatically illustrates an inner surface of a portion of an electronic garment according to another illustrative embodiment.

FIG. 8 diagrammatically shows an isolation view of a compression sleeve liner including a mixed ionic-electronic conducting (MIEC) material that may be worn underneath an electronic garment to improve electrical contact with skin.

FIG. 9 diagrammatically shows the compression sleeve liner of FIG. 8 worn on an arm.

FIG. 10 diagrammatically shows the worn compression sleeve liner of FIG. 9 with an electronic garment in the form of a sleeve disposed over the compression sleeve liner, with a portion of the electronic garment pulled back to reveal the inner surface thereof including electrodes disposed on the inner surface.

FIG. 11 diagrammatically shows the test setup for testing insulation of an electrically conductive thread with an electrically insulating coating.

FIG. 12 presents a measured EMG signal acquired using an electrode configured as shown in FIGS. 4 and 5 as described herein.

FIG. 13 presents measured impedance data for an electrode configured as shown in FIGS. 4 and 5 as described herein.

FIG. 14 diagrammatically shows various electrodes as described herein.

FIG. 15 presents impedance versus frequency (BODE) plots of different type of electrodes tested on surrogate skin (Test 1) as described herein.

FIG. 16 presents impedance measurements at 100 Hz (EMG frequency) (Test 1) as described herein.

FIG. 17 presents: (A) Impedance versus frequency (BODE) plot of the different type of electrodes on surrogate skin; and (B) zoomed-in on the y-axis of impedance versus frequency (BODE) plot of the different type of electrodes on surrogate skin. Testing was conducted at 20.1° C. and 40% Relative humidity (Test 2).

FIG. 18 presents interfacial charge transfer data of electrodes tested (Test 2) as described herein.

FIG. 19 depicts Table 1 which presents data as described herein.

FIG. 20 depicts Table 2 which presents data as described herein.

FIG. 21 presents normalized signal-to-noise ratio (SNR) data as described herein.

FIG. 22 presents normalized signal plots as described herein.

FIG. 23 presents SNR data with and without conductive spray of electrodes as described herein.

FIG. 24 presents interfacial charge transfer versus normalized SNR of tested electrodes (Test 2) as described herein.

### DETAILED DESCRIPTION

In some embodiments disclosed herein, a flexible electronic garment with a sleeve form factor (for example, sized and shaped to be worn on an arm, wrist, hand, leg, foot, or so forth) or other form factor provides a flexible, easy-to-use sleeve that comfortably maintains addressable electrodes in contact with the skin. The sleeve in some embodiments employs soft or flexible electrodes comprising a mixed ionic-electronic conducting (MIEC) material. Some suitable MIEC materials are described in Heintz et al., U.S. Pat. No. 11,305,106, which is incorporated herein by reference in its entirety. The electrodes including MIEC material provide good electrical contact with the skin even in the absence of a conductive gel. In some embodiments, the contact is improved using a conductive spray, such as Signa Spray. In some embodiments, the electrodes include an electrically conductive (e.g. metal) mesh such as a copper mesh, which provides good electrical conductivity in a highly flexible form factor. In some embodiments, the electrodes include an exposed electrically conductive thread, which provides good electrical conductivity in a highly flexible form factor. Such a copper mesh may, for example, be bendable up to 180°, i.e. turned back on itself. This provides a highly flexible electronic garment that also provides good electrical contact with skin. Such a sleeve or other wearable electronic garment may find various application, such as in sports rehabilitation, relaxation therapy, sports therapy, electrocardiogram (ECG) measurements, fetal scalp electrodes, electroencephalography (EEG) electrodes, functional electrical stimulation (FES), neuromuscular electrical stimulation (NMES), transcutaneous electrical nerve stimulation (TENS), haptic operations, electromyography (EMG), more generally electrophysiology measurements (e.g., ECG, EEG, EMG, et cetera), and so forth.

In various illustrative embodiments, medical apparatus and methods are disclosed, methods of manufacturing an MIEC-based textile, methods of treating a human or a nonhuman animal, a wound healing system, a method of administering a treatment; a wearable fabric such as a cuff or armband or sleeve; a method of recording or stimulating a nerve in a human or a nonhuman animal, a method of obtaining electrophysiology measurements (e.g. an EEG, ECG, or EMG or so forth), comprising utilizing one or more features of the invention and an electrode, in some embodiments without use of a hydrogel. Illustrative embodiments are also directed to methods of making a wearable garment, comprising: providing an elastic fabric; attaching the metal (e.g. copper or copper alloy) mesh/thread to the fabric, or a variant for carbon nanotube fiber/textile as the conductive mesh/thread (e.g. by sewing); applying an MIEC precursor composition onto the electrically conductive mesh/thread; and curing the MIEC precursor composition. Various illustrative embodiments are also directed to methods of treating a human or nonhuman subject comprising applying the MIEC composite to the skin of the subject and applying a potential.

Various embodiments disclosed herein can provide various advantages such as one or more of the following advantages: one-part electrode system for electrical stimulation; easy to don and doff (less than 10 seconds in some cases); an electronic garment that conforms to skin and reduces likelihood of electrical arcing during electrical stimulation of anatomy on which the electronic garment is worn using the electronic garment; increased breathability of an electronic garment; decrease in weight of an electronic garment (by way of nonlimiting illustrative example, for a sleeve using a copper mesh instead of stainless steel button, and with 160 electrodes, the weight of only the current collector is reduced from 64 grams down to 2.64 grams); improved the overall performance (or reaction time) of the garment since the body is not weighed down by a heavy electronic garment; easy washability of an electronic garment; improved durability/longevity of components by eliminating exposed bare metal which can corrode over time; and/or so forth.

In various embodiments, an integral, conforming electronic garment for electrical stimulation and/or reading of electrophysiology signals (e.g., EMG) is disclosed. A conductive thread provides an electrical connection to a conductive mesh that connects to an elastic material that comfortably forms an electric connection to a person's skin. This can be made by taking an electrically conductive thread (for example, nylon with a silver plated thread, carbon nanotube fiber or copper wire) and stitching it (e.g. with couch stitching, a zig-zag pattern, or so forth), with a durable non-conductive thread on an elastic textile such as spandex to secure the conductive thread to the top layer of the textile. A dielectric layer is coated around the wire (outside of the electrode area) to eliminate arcing between threads and biting for the wearer of the electronic garment. A liquid metal (such as a Gallium alloy) could also be used as a connector instead of an electrically conductive solid wire/thread. A section (such as 4 cm in length) of bare conductive thread (with the coating dielectric layer removed) can be stitched against a circular copper mesh fabric or stitched in any form factor on the fabric without the metal mesh; this acts as the electronic conductor. The copper mesh (or other electrically conductive mesh material) can have a chosen form factor for the application and each mesh forms one electrode. To provide good electrical contact with skin, a liquid precursor, such as an MIEC slurry, is then deposited on and through the mesh and cured to create an electrode. The conductive thread is then connected to a stimulation hub to allow for voltage and current to flow through it to induce electrical stimulation.

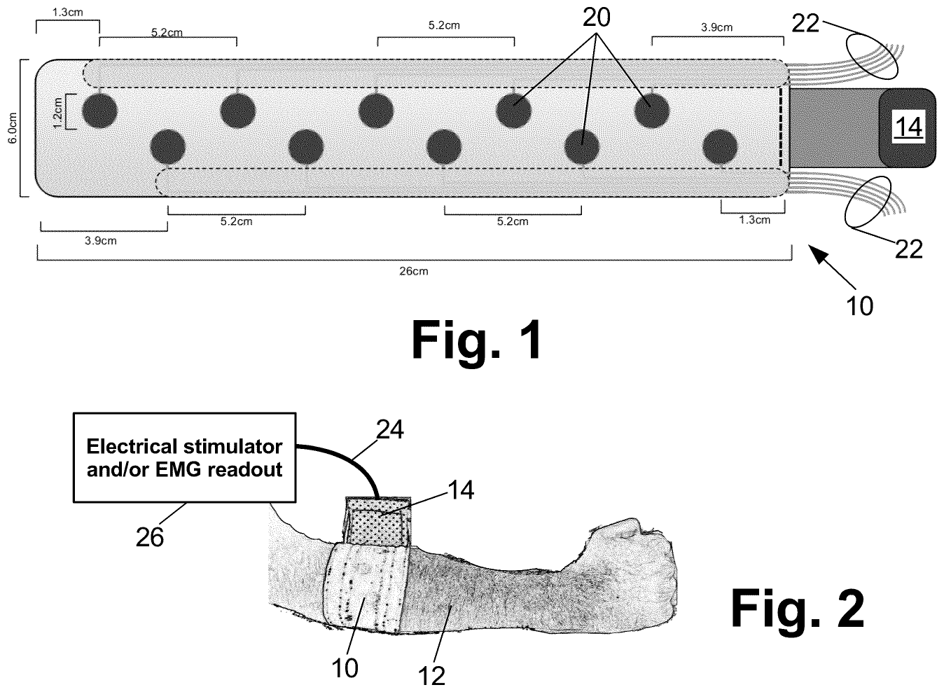

With reference to FIGS. 1 and 2, an illustrative example of an electronic garment 10 is shown. The electronic garment 10 is suitably made of a woven fabric or the like, which in some embodiments is an elastic fabric such as Spandex™, Lycra™, elastane or so forth comprising synthetic fibers with high elasticity making the electronic garment 10 as a whole elastic. The illustrative electronic garment 10 is armband which is shown unwrapped in FIG. 1 and wrapped around the forearm 12 of a wearer in FIG. 2 and secured around the forearm by a Velcro™ fastener 14 or the like. It is to be understood that this is merely a nonlimiting illustrative example, and that more generally the electronic garment 10 could have the form factor of an armband, wristband, leg band, a sleeve covering part or all of the arm and/or wrist and/or hand, a sleeve covering part or all of a leg and/or ankle and/or foot, a vest covering the torso, a skullcap, various combinations thereof, and/or so forth. In such various types of electronic garments, the mechanism for securing or fastening the garment to the anatomy of the wearer can be various, e.g. an electronic garment in the form of a sleeve can be an elastic sleeve in which fabric of the sleeve is elastic and the sleeve is held on the arm or leg or other anatomy by the elastic sleeve compressing against the arm. Some further examples of suitable form factors of wearable electronic garments are described in Bouton et al., U.S. Pat. No. 9,884,178 issued Feb. 6, 2018 and Bouton et al., U.S. Pat. No. 9,884,179 issued Feb. 6, 2018, both of which are incorporated herein by reference in their entireties. Again, these are merely nonlimiting illustrative examples.

As shown in FIG. 1 which shows the unwrapped armband 10 viewing the interior side of the armband that contacts the skin of the forearm 12, the armband includes a set or plurality of electrodes 20 connected with insulated electrically conductive threads 22 that port electrical signals (current and/or voltage) to and/or from the electrodes 20 of the electronic garment 10. When the electronic garment 10 is worn on anatomy 12 of the wearer (e.g. as shown in FIG. 2), the electrodes 20 contact skin of the anatomy 12 of the wearer. The illustrative insulated electrically conductive threads 22 are arranged as two peripheral buses on opposite sides of a central region of the armband 10 containing the electrodes 20; however, it will be understood that the routing of the insulated electrically conductive threads 22 can be various, e.g. the insulated electrically conductive threads 22 may be routed through gaps between electrodes or so forth. The illustrative example of FIG. 1 includes some nonlimiting illustrative dimensions for the electrodes 20 and the separations between electrodes 20 along various directions—again, these are to be recognized as a nonlimiting illustrative example.

As diagrammatically shown in FIG. 2, the insulated electrically conductive threads 22 are wired to a pigtail or bundler or other cable 24 that connects with electronics 26 such as an electrical stimulator for neuromuscular electrical stimulation (NMES) or transcutaneous electrical nerve stimulation (TENS) or functional electrical stimulation (FES) or so forth, and/or to electronics 26 such as an electromyography (EMG) amplifier (or other electrophysiology measurement amplifier) and readout electronics. In some designs, a portion of the electronics 26 may be integrated with the electronic garment 10 itself (variant not shown), for example as electronic cards or modules embedded in or attached to into the fastener 14.

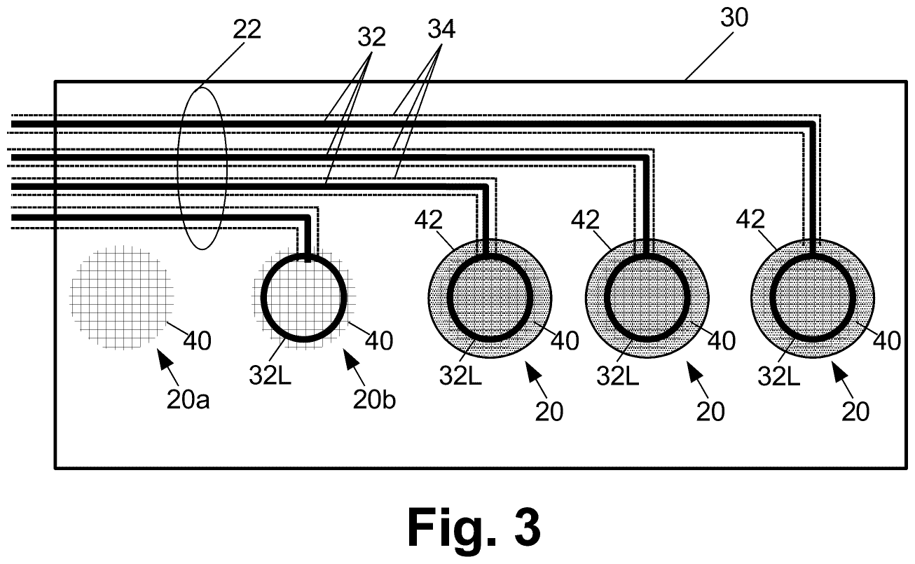

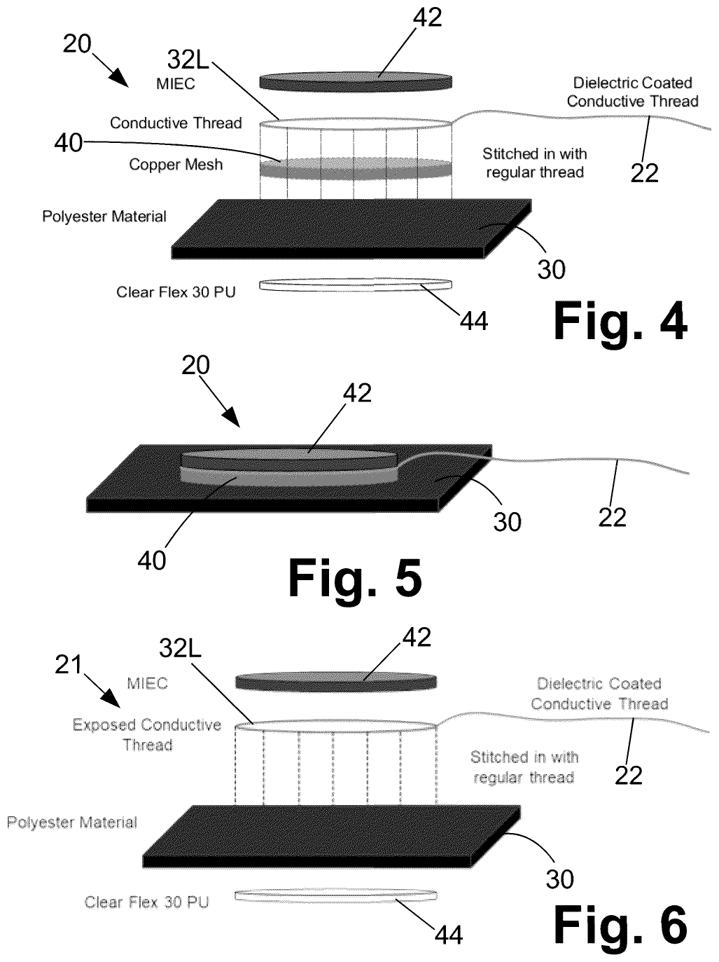

With reference now to FIGS. 3-5, some suitable embodiments of the electrodes 20 are described. FIG. 3 illustrates the inner surface (that is, the surface that contacts the skin when worn on anatomy) of a portion of an electronic garment (which could, for example, be a portion of the electronic armband 10 of FIGS. 1 and 2 as one nonlimiting illustrative example). The portion of the electronic garment shown in FIG. 3 includes a portion of the Spandex™ or other electrically nonconductive fabric 30 making up the electronic garment along with three electrodes 20 and two partially formed electrodes 20a and 20b. The partially formed electrodes 20a and 20b are shown for illustrative purposes, and it will be understood that in the finally formed electronic garment all electrodes are typically completely formed electrodes. The insulated electrically conductive threads 22 are shown in FIG. 3 as including an electrical conductor (e.g. wire) 32 coated by an electrically insulating coating 34. Where each insulated electrically conductive thread 22 connects with the electrode 20, the electrically insulating coating 34 is removed so that it does not interfere with electrical connection to the electrode 20. In the illustrative example of FIG. 3, an exposed end 32L of the insulated electrically conductive thread 22 has the insulation 34 stripped to expose the electrical conductor (e.g. wire end) 32L which in the illustrative example is formed into a loop 32L that is generally aligned with the (illustrative circular) area of the electrode 20; however, other arrangements for this connection 32L are contemplated, including omitting the loop geometry and running a straight (or approximately straight) stripped end of the insulated electrically conductive thread 22 onto the electrode 20. More generally, there is an electrically exposed portion 32L of the insulated electrically conductive thread 22. FIG. 4 illustrates an exploded perspective view of one electrode 20, and FIG. 5 shows a perspective view of one electrode 20. As best seen in FIG. 4, the electrode 20 includes: a metal mesh 40, such as an illustrative copper mesh 40, that is woven into or otherwise attached to the electrically nonconductive fabric 30 of the electronic garment; and the stripped end 32L of the insulated electrically conductive thread 22 which is secured for example by couch stitching the loop 32L to the underlying electrically nonconductive fabric 30 (note that FIGS. 4 and 5 omit illustration of the insulation 34 shown in FIG. 3). The electrode further includes, or viewed alternatively is in contact with, an electrically conductive polymer electrode material 42 that is disposed on the metal mesh 40 and/or fabric 30. In the illustrative embodiment, the electrically conductive polymer electrode material 42 comprises a mixed ionic-electronic conductive (MIEC) material 42 that is disposed on the metal mesh 40 and/or fabric 30. FIG. 3 diagrammatically illustrates one approach for manufacturing such an electrode 20 by way of the partially fabricated electrodes 20a and 20b. The partially fabricated electrode 20a has only the metal mesh 40 woven into or secured with the fabric 30 of the electronic garment. The partially fabricated electrode 20b has the metal mesh 40 woven into or secured with the fabric 30 of the electronic garment and also has the stripped end 32L of insulated electrically conductive thread 22 secured to the electrode-under-fabrication by couch stitching, zig-zag stitching, or the like. The remaining three completed electrodes 20 shown in FIG. 3 include the metal mesh 40 woven into or secured with the fabric 30 of the electronic garment and electrically exposed portion 32L of insulated electrically conductive thread 22 secured by couch stitching, weaving, or the like, and also include the deposited MIEC material 42. In the illustrative example of FIG. 3, the MIEC material 42 extends over the entire metal mesh 40 and also extends a small distance beyond the boundary of the metal mesh 40; however, in other embodiments the MIEC material may be coextensive with the metal mesh, or may be disposed over only a central portion of the metal mesh. However, having the area of the MIEC material 42 be larger than the metal mesh 40 as shown, or at least coextensive therewith, can reduce likelihood of electrical arcing at an uncovered portion of the metal mesh when electrical stimulation is applied to the skin using the electrode 20.

Some suitable MIEC materials for use as the MIEC material 42 are described in Heintz et al., U.S. Pat. No. 11,305,106, which is incorporated herein by reference in its entirety. As there described, mixed-ionic-electronic conductors comprise an interconnected network of electrical and ionic conductors in an elastomeric matrix that provide high surface area for capacitive charge-discharge and high ionic conductivity for low interfacial charge transfer. MIEC materials provide low ohmic resistance and good flexibility and toughness. In the MIEC material, electrical and ionic conductors are embedded in a matrix in such a way that the electrical and ionic elements achieve percolation, i.e., a continuous interconnected network, at lower loading than would be achieved by random mixing. This allows superior electrical performance to be achieved while retaining good mechanical properties. The morphology of the MIEC material can be controlled by using a polymer latex, also called an emulsion, in which polymer particles are dispersed in an aqueous phase, to template the organization of the electrical and ionic conductors. Examples of suitable dispersions include elastomeric polymers such as nitrile butadiene rubber, natural rubber, silicone, Kraton-type, silicone acrylic, or polyurethane. Other suitable polymer lattices include polyvinylidene fluoride or polyvinylidene chloride. In such a dispersion, at least 90 mass % of the polymer particles are preferably in the range of 50 nm to 10 μm in diameter. The dispersion is cast and the volatiles (e.g., water) allowed to evaporate. During evaporation, the polymer particles coalesce to form a continuous fill. The electrical and ionic conductors are added to the latex so that they are dispersed in the aqueous phase. The pH may be balanced, and dispersing agents can also optionally be used. Some suitable electrical conductors for the MIEC material include electrical conductors that have high aspect ratio and are readily dispersed into aqueous solutions and include carbon nanotubes, graphene and graphite structures, and metal nanowires. Suitable ionic conductors include sodium hyaluronate, also called hyaluronic acid, fluorosulfonic acids like Nafion™, sulfated polysaccharides and other mucoadhesive type compounds, or other phosphonic polyvinylsulfonic acids. Likewise, anisotropic ionic conductive particles like graphene oxide and modified graphene oxide may be used. In some embodiments, hyaluronic acid is used due to its advantageous tendency to hydrate with the skin, thereby improving skin contact. By adding electrical and ionic conductors to the dispersed phase of the latex, the conductors tend to coat the surface of the polymer particles, but not penetrate. As the latex is dried, the conductors tend to be confined at the interfaces, creating an interconnecting network, where the major phase is elastomeric and a connected thin, layer phase is the electronic/ionic conductors. The morphology of this network can be modified by changing the particle size of the polymer in the latex. Larger particle sizes require less conductor to reach an interconnected phase. The film formation temperature is also a tunable parameter that can used to modify the kinetics to achieve various kinetically trapped states. Other methods to achieve better than random mixing include self-assembling or self-stratifying coatings.

In some nonlimiting illustrative embodiments, the MIEC material includes carbon nanotubes which are the electrical conductors; and hyaluronic acid, or other glycosaminoglycan, along with moisture and ions, serve as the ionic conductor. In some embodiments, the MIEC material has high conductivity of at least 1000 mS/cm, and in some embodiments at least 2000 mS/cm, and in still other embodiments electrical conductivity in the range of 2000 mS/cm to about 4000 mS/cm. The MIEC material may have high moisture retention such that the composite may absorb at least 20% water, up to 50% by mass water (corresponding to 100% of the weight of the dry composite), in some embodiments 20% to 50%, or 35% to 50% water.

In some embodiments, carbon nanotubes are the electrical conductors and hyaluronic acid (HA), or other glycosaminoglycans, along with residual atmospheric moisture and ions, is the ionic conductor. In some embodiments, the MIEC material comprises 0.1 to 2 wt % CNTs, including 0.2 to 1 wt %, and in some embodiments 0.5 to 0.8 wt % CNTs (by weight of the as dried MIEC material). In some embodiments, the MIEC material comprises 0.1 to 5 wt % glycosaminoglycan, for example in a range of 0.4 to 4 wt %, and in some embodiments 0.7 to 3 wt % glycosaminoglycan. In some embodiments, the as-dried mass ratio of glycosaminoglycan to CNT in the MIEC material is in the range of 0.5 to 105, preferably 41 to 83, and in some embodiments 1.5 to 2.5. In some embodiments, the MIEC material comprises at least 0.01 wt % Na, or 0.01 to 2 wt % Na, and in some embodiments 0.1 to 1 wt % Na. This may occur, for example, when the ionic conductor is sodium hyaluronate. It is further contemplated that the MIEC material can be characterized by any one or any combination of these properties. The remainder of the MIEC material is formed from the elastomeric phase.

In some nonlimiting illustrative embodiments the MIEC material 42 of (or in contact with) the electrode 20 can be viewed as having a top and bottom surface, in which the bottom surface is adapted to contact the skin of a patient, and the electrode has a graded structure with an increasing ratio of ionic conductor to electrical conductor from the top to the bottom of the electrode. The gradient is prepared by layer-by-layer fabrication of the electrode, with increasing levels of ionic conductor in successive layers; in some embodiments having at least 3 layers or at least 5 layers. The elastomeric particles may comprise nitrile butadiene rubber, natural rubber, silicone, Kraton-type, silicone acrylic, polyvinylidene fluoride, polyvinylidene chloride, or polyurethane, or combinations thereof. In the emulsion prior to curing, at least 90 mass % of the polymer particles are in the size range of 50 nm to 10 μm in diameter. The electrical conductors have a number average aspect ratio of height to the smallest width dimension of at least 10. The electrical conductor may comprise carbon nanotubes, graphene, graphite structures, metal nanowires, or various combinations thereof. The ionic conductor may comprise hyaluronic acid, a fluorosulfonic acid like Nafion™, sulfated polysaccharides and other mucoadhesive type compounds, or other phosphonic polyvinylsulfonic acids, or various combinations thereof. The polymeric or elastomeric polymer may comprise an adhesive polymer or wherein the electrode further comprises an adhesive polymer. The coalesced polymeric particles may comprise a fluoropolymer.

The carbon nanotubes (CNTs), if included in the MIEC material 42, may be single, double, and multiwall CNTs, and may optionally also include bundles and other morphologies. The CNTs can be any combination of these materials, for example, a CNT composition may include a mixture of single and multiwall CNTs, or it may comprise double-walled CNTs (DWNT) and/or multiwalled CNT's (MWNT), or it may comprise of single-walled CNT's (SWNT), various combinations thereof, or so forth. The CNTs of the MIEC material 42 may in some embodiments have an aspect ratio (length to diameter) of at least 50, preferably at least 100, and in some embodiments more than 1000. In some embodiments, a CNT network layer is continuous over a substrate; in some other embodiments, it is formed of rows of CNT networks separated by rows of polymer (such as CNTs deposited in a grooved polymer substrate). The CNTs may be made by methods known in the art such as arc discharge, CVD, laser ablation, or HiPco.

In the above example, the MIEC material 42 is fabricated as one or more layers (e.g. 3-5 layers) of MIEC material. However, the MIEC material 42 may be otherwise applied. For example, in another embodiment the MIEC material 42 comprises a fabric impregnated with MIEC material. Such a fabric can be sewn into the garment 10 over the electrically conductive mesh 40, for example. Still further, while a MIEC material such as described above is suitable as the electrically conductive polymer electrode material 42 disposed on the electrically conductive mesh 40, in other embodiments the electrically conductive polymer electrode material 42 may be another type of electrically conductive polymer electrode material 42, such as an electrically conductive hydrogel material, which is a crosslinked hydrophilic polymer that does not dissolve in water.

In the following, some further embodiments of the electrodes 20 are described.

The electrically nonconductive fabric 30 of the electronic garment may, for example, comprise an elastic polyester material such as Spandex™, Lycra™, elastane or so forth. In some nonlimiting illustrative embodiments, the fabric 30 comprises a polyether-polyurea copolymer mixed with other synthetic or natural fibers such as cotton. These are merely nonlimiting illustrative examples.

The electrically conductive mesh 40 (e.g. illustrative copper or copper alloy mesh or other electrically conductive mesh material) is sewn into the elastic textile 30 making up the electronic garment to provide an electrical connection between the electric current (or voltage) source and/or measurement device 26 (see FIG. 2) via the insulation-coated electrically conductive thread 22. The mesh can be provided in any shape and/or size, with a circular shape being illustrated. The conductive mesh 40 should be a flexible mesh to provide good flexibility for the electronic garment. The metal wires or other electrically conductive wires of the conductive mesh 40 are, in one nonlimiting illustrative example, in the size range of 0.05 to 0.5 mm or 0.1 to 0.3 mm or 0.1 to 0.2 mm. The conductive mesh 40 can have a wide range of mesh sizes (e.g., quantified by openings per square inch), such as a 50 to 500 mesh or 80 to 300 mesh in some nonlimiting illustrative embodiments. The conductive mesh 40 in one embodiment comprises copper or a copper alloy, e.g. at least 90% copper in some embodiments, or at least 95% copper in other embodiments, or at least 99% copper in still other embodiments. The conductive mesh 40 is suitably secured to the electrically nonconductive fabric 30 of the electronic garment by a conductive thread (or in other embodiments, an electrically nonconductive thread) by knitting, especially knitting around the periphery of the mesh.

In a variant embodiment, the electrically conductive mesh 40 can be replaced by another type of flexible metal layer, such as a thin metal sheet, such as a copper or copper alloy sheet, or a carbon nanotube fiber/textile conductive mesh, or so forth. In a variant embodiment employing a thin metal sheet, the thin metal sheet should be thin enough to enable a sewing needle to penetrate through the sheet to secure it to the electrically nonconductive fabric 30 by sewing or the like. Connection of the electrically exposed portion 32L of the insulated electrically conductive thread 22 to the flexible metal layer can likewise be by sewing as described for the embodiments in which the flexible metal layer is a metal mesh, or can be by a method such as welding, soldering, or another type of metal joining. In some embodiments, the thin copper or other metal sheet is thin enough to allow for it to be capable of a full 180° fold back upon itself, providing a high degree of flexibility.

The mesh 40 sewn into an elastic textile corresponds to the partially fabricated electrode 40a shown in FIG. 3. Thereafter, the electrically exposed portion 32L of the insulated electrically conductive thread 22 is secured to the mesh 40 by couch stitching or the like, thereby producing the partially fabricated electrode 40b of FIG. 3. Thereafter, a liquid precursor to an electronic conductor is deposited onto the conductive mesh, such as an MIEC slurry, and is cured to form the MIEC material 42. FIGS. 4 and 5 show the MIEC material 42 as a distinct layer disposed on the copper mesh 40; however, as the MIEC slurry may flow through openings of the metal mesh 40 and may infuse into the fabric 30, the resulting MIEC material 42 may in some embodiments be at least partly disposed in openings of the metal mesh 40 and possibly also may be infused into the underlying fabric 30 of the electronic garment. A shown in FIG. 4, optionally the backside of the electrode 20 can be coated with polyurethane or another insulator 44 (e.g., Clear Flex 30 polyurethane in illustrative FIG. 4) to eliminate any biting (e.g., electrical discharge through the backside of the electronic garment at the location of the electrode 20) if someone were to touch the backside during electrical stimulation using the electrode 20. In some testing no such biting was observed from the backside, but with the addition of an optional conductive spray such as Signa spray, or in the presence of perspiration from the wearer, some biting could potentially occur, so that the backside coating 44 is an optional precautionary step.

Some nonlimiting illustrative examples of the insulated electrically conductive thread 22 are as follows. Conductive yarns can be made with conductive strands woven into a yarns, e.g. with nonconductive fibers, and/or nonconductive fibers with conductive coatings. An electrically conductive yarn forming the electrically conductive core 32 of the insulated electrically conductive thread 22 (see FIG. 3) can be encased in a dielectric material, such as an enamel and polymeric insulative coating, to form the electrically insulating coating 34.

To secure the insulated electrically conductive thread 22 to the fabric 30 of the electronic garment, various approaches can be used, such as couch stitching or three-dimensional (3D) knitting, so as to enable routing a large number of such threads 22 to the electrodes 20. In general, if there are N electrodes 20 then N threads 22 connecting to the N respective electrodes 20 are used to provide fully individualized addressing of the N electrodes. For some applications such as high-density EMG, well over 100 electrodes may be included in an electronic garment with a sleeve form factor. Conductive yarns forming the insulated electrically conductive threads 22 can be three dimensionally knitted using an industrial 3D knitting machine, machined, woven, or hand knitted (optionally along with non-conductive thread into any textile form factor, not limited to a swatch, band, headband, shirt, pants, socks, sleeve, etc. This can be accomplished by knitting the insulated electrically conductive threads 22 intertwined into non-conductive yarn/threads of the fabric 30, with uninsulated portions 32L of the insulated electrically conductive thread 22 woven into exposed electrode patches 20 to interface with the conductive polymer (e.g., MIEC material 42). A conductive polymer precursor can be cured into and/or on the exposed, uninsulated thread regions, to create electrodes. The nonconductive threads can be a “dry fit,” polyester material, for example.

A 3D knitting machine can make precise stitches of the insulated electrically conductive threads 22 with the dielectrics layers 34 to produce an electronic garment 10 with high electrode density. The 3D knitting machine can be used with a conductive yarn with the dielectric layer to form the insulated electrically conductive thread 22 along with other electrically nonconductive yarns for forming the fabric 30—in this way, the entire electronic garment 10 can in some embodiments be constructed as a single piece. Advantageously, in this approach there is no need to sew in the conductive yarn with the dielectric layer (i.e., the threads 22) onto a commercial off-the-shelf (COTS) textile material.

To form the stripped ends 32L of the insulated electrically conductive threads 22 for connection to the electrode 20, the dielectric layer 34 and/or materials can be removed, for example by burning, melting, acid etching, or the like, to leave sections 32L with the conductor 32 exposed or only of the metallic conductor 32, allowing the polymeric conductor material to be anchored onto the exposed section. This allows for one complete system, eliminating the need for a flex circuit, a separate current and ionic collector, hydrogels, conductive lotions, and multiple parts.

Benefits of the knitting or weaving directly into a textile form factor include one or more of the following: decrease in total weight of garment; increased washability; increased flexibility; easy to don and doff (less than 10 seconds); increased breathability; increased conformability; superior performance of the sleeve since the arm will not be weighed down as much; one-piece electrode system for electrical stimulation; flexible stretchable electrode; and increased elasticity.

With reference to FIG. 6, a variant embodiment of an electrode 21 is diagrammatically shown in the exploded view. The electrode 21 is similar to the electrode 20 of FIGS. 4 and 5 but which omits the electrically conductive mesh 40 of the electrode 20 of FIGS. 4 and 5. Hence, the electrode 21 includes the electrically exposed portion 32L of the insulated electrically conductive thread 22 which is sewn (or weaved) onto or into the elastic textile garment 30. Here the conductive polymer (e.g., MIEC material 42) is disposed on the elastic textile garment 30 in electrical contact with the electrically exposed portion 32L of the insulated electrically conductive thread 22. The backside of the electrodes 20 can optionally be coated with polyurethane (or other insulator) 44 as previously described with reference to FIG. 4.

In the embodiments described with reference to FIGS. 3-6, MIEC material 42 is deposited individually on each individual electrode 20.

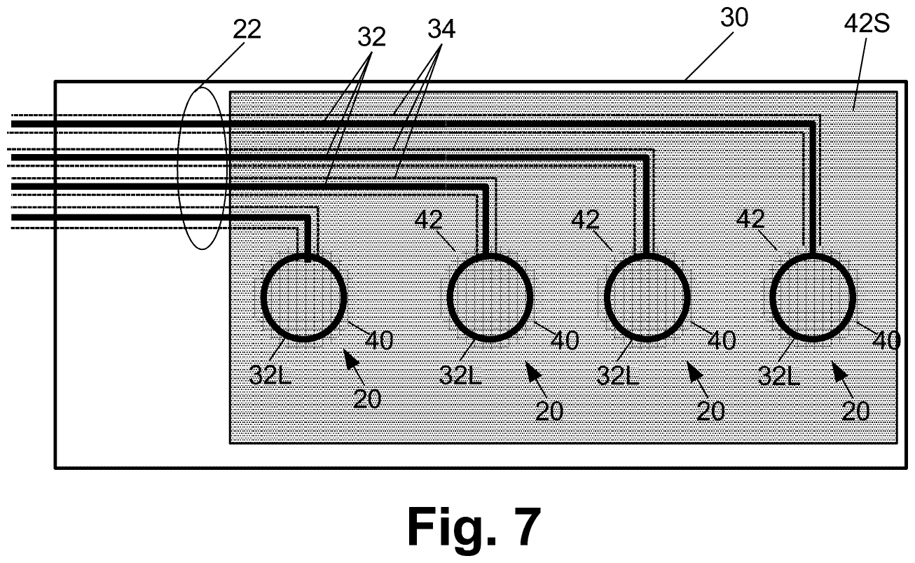

With reference to FIG. 7, in other embodiments it is contemplated to employ a thin sheet of MIEC material that extends over multiple (possibly all) of the electrodes 20 of the electronic garment. This is shown in FIG. 7, which is similar to FIG. 3 except that the individual regions of MIEC material 42 of FIG. 3 are replaced by an MIEC sheet 42S comprising the MIEC material. As long as the sheet is sufficiently thin, electrical conduction between neighboring electrodes 20 through the MIEC sheet 42 is avoided. In one approach, the MIEC sheet 42S can be secured with the fabric 30 of the electronic garment. For example, the MIEC sheet 42S can comprise an MIEC slurry cured in a mold. In other embodiments, the MIEC sheet 42S can comprise a thin fabric coated and/or infused with MIEC material, which is then sewn to the fabric 30. This latter approach effectively forms a two-ply garment, including the electrically nonconductive fabric 30 as one ply and the MIEC sheet 42S as the second ply, with the metal meshes 40 forming the electrodes 20 and the insulated electrically conductive threads 22 disposed therebetween and sewn to the electrically nonconductive fabric 30.

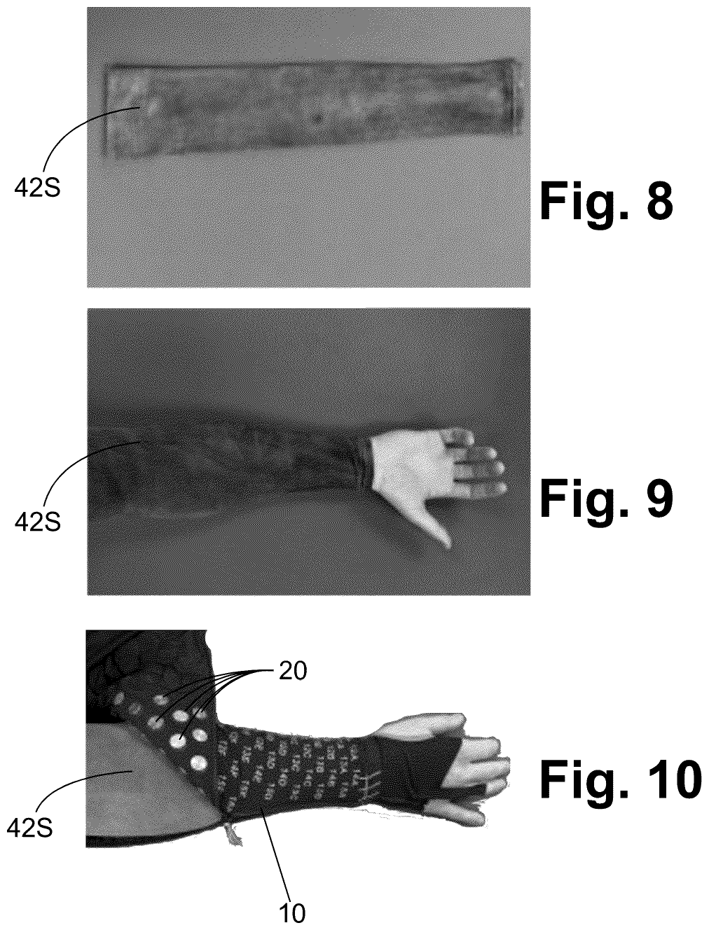

With reference to FIGS. 8-10, in other embodiments the MIEC sheet 42S is embodied as a separate compression sleeve or the like, which is disposed between the skin and the electrode sleeve. FIG. 8 shows an isolation view of such an MIEC sheet 42S in the form of a separate compression sleeve liner. The compression sleeve liner 42S of FIG. 8 is a garment that when worn on target anatomy compresses against that target anatomy of the body, which is coated and/or infused with MIEC material. This is shown in FIG. 9, where the MIEC sheet 42S in the form of a compression sleeve liner is worn on an arm. The liner 42S is elastic and does not rip easily. In one nonlimiting illustrative embodiment, the MIEC sheet 42S is in the form of a compression sleeve liner comprises 88%-92% nylon/polyamide/polyester fibers and 8%-12% spandex. FIG. 10 shows the MIEC sheet 42S in the form of a compression sleeve liner, with the electronic garment 10 in the form of a sleeve disposed over the liner 42S, with a portion of the electronic garment 10 pulled back to reveal its inner surface with the electrodes 20.

The compression sleeve liner 42S can be impregnated with a precursor to a conductive elastic composition, e.g. an MIEC material precursor, and then cured. This produces a compression sleeve liner 42S that is coated and/or infused with MIEC material. In experiments, it was found that the resulting MIEC compression sleeve liner 42S suppressed burning/biting from lifting electrodes and allows for more localized electrical stimulation, and also increased the signal to noise ratio (SNR) for EMG recording when compared to the use a non-compressive sleeve. The testing was conducted using an impregnated compression liner as a conduction enhancer. With this setup, EMG signal was successfully recorded across 70 channels of an electronic garment in the form of an EMG Sleeve. Notably, the electrical stimulation was significantly more localized when using the MIEC compression sleeve liner 42S comprising a fabric impregnated with MIEC material, as compared to using an MIEC sheet 42S that is an MIEC slurry cured in a mold. In other words, when the MIEC sheet 42S comprises a fabric that is infused or impregnated with MIEC material, more of the electric current travels between the electrodes and the skin (rather than between neighboring electrodes) than is the case when the MIEC sheet 42S comprises an MIEC slurry cured in a mold. Without being limited to any particular theory of operation, it is believed that the discrete nature of the interwoven threads of the MIEC-infused fabric increases the in-plane resistance to electrical current flow across the MIEC sheet; whereas, the cured MIEC slurry provides lower electrical resistance in the plane of the sheet. The compression sleeve liner design comprising MIEC-infused fabric thus provides more accurate targeting of the correct muscles for the correct range of motion. Feasibility testing was conducted using the liners and participants were able to evoke muscle movement at similar currents to that of a hydrogel sheet. The participants indicated that the electrical stimulation felt more focused and less spread out, allowing for more intricate stimulation movements. Hence, the MIEC compression sleeve liner 42S provides similar current transfer efficiency to skin as a hydrogel sheet, but with better spatial resolution. As another advantage, the MIEC compression liner 42S allows for a single person to don and doff an electrode-containing electronic sleeve. Thus, advantages include: localized electrical stimulation; higher SNR for EMG recording; superior anchoring to a patient's arm; better mechanical durability than MIEC sheets due to added strength of the woven fiber liner.

In some embodiments, a conductive spray such as Signa™ spray can be applied to the skin and/or directly to the inner surface of the garment 10 prior to donning the electronic garment 10 to further improve electrical contact with the skin. A downside of conductive spray, as well as some MIEC materials, is that they can have an odor that some find disagreeable. In some embodiments, a scent is added to the wearable fabric 30 or the conductive spray, such as Signa™ spray. For example, by adding an ester to the fabric 30 and/or to a conductive spray applied to the skin before donning the electronic garment, the odor of the fabric or spray can be improved. The benefit of adding a scent to the conductive spray allows for a more natural feeling when using an electrode that smells artificial. This will eliminate the smell of rubber and the bad smell of hydrogel, providing a more positive experience for the wearer of the electronic garment as it will eliminate the smell of rubber, the hydrogel smell, and the smell of other sticky electrodes. The spray could be sprayed onto the MIEC sheet as well as the users targeted body part. Some suitable esters and their scents are as follows:

TABLE 1

| Ester | Scent |
|---|---|
| allyl hexanoate | Pineapple |
| benzyl acetate | Pear |
| butyl butanoate | Pineapple |
| ethyl butanoate | Banana |
| ethyl hexanoate | Pineapple |
| ethyl heptanoate | Apricot |
| ethyl pentanoate | Apple |
| isobutyl formate | Raspberry |
| isobutyl acetate | Pear |
| methyl phenylacetate | Honey |
| nonyl caprylate | Orange |
| pentyl acetate | Apple |
| propyl ethanoate | Pear |
| propyl isobutyrate | Rum |

A useful attribute of the disclosed flexible electrodes 20 that include the electrically conductive mesh 40 and MIEC material 42 is its application to various wearable and comfortable forms, such as foam or fabric. No hydrogel is necessary to couple to the skin. Mechanical contact can be provided by applying an elastomeric band around the material or using an elastomer or adhesive as the polymer, or using an elastic sleeve form factor for the electronic garment or so forth. This approach avoids motion artifacts in EMG recording electrodes due to squeeze out of the hydrogel.

Peripheral nerves can be stimulated to treat neural disease. However, most nerve stimulating interfaces are implanted using an invasive surgery. To overcome this short coming, non-invasive nerve stimulation can be implemented using a hydrogel or other ‘wet’ conductive interface to transcutaneously stimulate nerves at a reasonable depth below the skin. Unfortunately, this ‘wet’ electrode—skin interface is suboptimal for long term peripheral nerve stimulation (hours—days). Furthermore, shifts in ‘wet’ electrodes over time interfere with therapeutic efficacy and the location of applied current fields below the skin. The disclosed electrodes 20 that include the electrically conductive mesh 40 and MIEC material 42 provide an interface that is capable of long term current steered non-invasive peripheral nerve stimulation to treat neural or non-neural disease. Any peripheral nerve that can be reliably activated transcutaneously (that is, through the skin) and affect physiological function is a candidate for being stimulated using the electrodes 20 disclosed herein. For example, peripheral nerve stimulation-based therapies to treat disease (e.g., auricular nerve stimulation for atrial fibrillation or trigeminal nerve stimulation for migraine) are some nonlimiting illustrative applications. The nerves that can be specially targeted for non-invasive nerve stimulation are many, and some are listed above for example cases.

With returning reference now to FIGS. 1-5, some actually performed experiments are described. A prototype was constructed to test the feasibility of the disclosed electrodes 20, which employed the armband 10 of FIGS. 1 and 2. The armband 10 was made with 2 rows of 5 electrode (10 total electrodes). This device is referred to herein as a “Flex NeuroBand,” and was constructed by first coating the conductive threads 32 with a dielectric layer to form the insulation 34. This was completed so that there would be no exposed conductive thread, as any exposed conductive thread could electrocute/burn/bite the skin. Various dielectric materials can be used for this applied insulation 34; in the experiments reported here ZEON Nipol LX370 was applied, which is the same NBR material that we use when formulating the MIEC slurry which was subsequently cured to form the MIEC material 42. More generally, any suitable polymer based dielectric can be used to coat the conductive thread 32 to form the insulation 34. The setup for coating the conductive threads was as follows: the conductive threads were hung from a rode in a hood separated by about 4 inches from one another. About 20 inches of each thread was coated with the Nipol LX370 material. Next, a 2 mL pipet was filled with the Nipol LX370 material and it was deposited at the top of the conductive thread and allow for gravity to flow the NBR smoothly down the thread. Each drop was considered one layer. Each thread was coated 30 times, with a wait period of 10 minutes after every 5 coats (6 coats of 5 repetitions). Once all the coats were finished, the threads were placed in an oven at 50° C. for 2 hours to cure the insulation 34. The thickness of the conductive thread 32 with the dielectric layer 34 thus manufactured was around 0.7 mm.

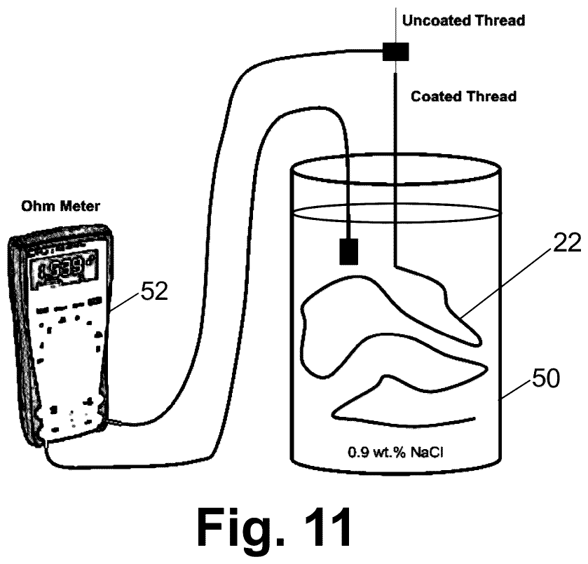

With brief reference to FIG. 11, a quality assurance check was then conducted on the threads to ensure that there was no exposed conductive thread. FIG. 11 shows the test setup, in which the insulation-coated thread 22 was disposed in a container 50 filled with an electrolyte (0.9 wt. % NaCl solution) and an ohmmeter 52 was used to test for an electrical current. Fully coated threads exhibited 0 amps. (Any gap in the insulation 34 would provide an ingress path for electrical current from the electrolyte to the electrically conductive (e.g. metal) core of the insulation-coated thread 22).

Once the insulated electrically conductive threads 22 were thus fabricated and tested, an end of each thread was stripped of insulation to form 4 cm of bare conductive threads which will form the end 32L shown in FIGS. 3 and 4. In parallel, a sheet of copper mesh (100 mesh from TWP Inc) was cut to form the copper meshes 40 of the electrodes 20 as disks of 12 mm diameter each. The exposed side 32L of the conductive thread was placed over top and in direct contact of the copper mesh 40 along the circumference and sewn around the circumference of conductive thread and copper mesh to attach it on a polyester matrix (dry fit/spandex material, corresponding to the fabric 30 of FIGS. 3-5) with a nonconductive thread such as cotton or polyester. A tear away sewing stabilizer was used on the backside of the polyester matrix to allow for more accurate and easier sewing to ensure the electrode diameter is maintained. The stabilizer was removed after sewing was completed. In more detail: Make sure the insulated conductive threads are side by side and make a few stiches over them. Place a piece of polyester matrix over the threads and sew it on by a technique called couch stitching. Once the stitching is complete, take vinyl stickers and cut out holes to the appropriate size of the electrodes. These stickers act as a template/well for the drop casted MIEC slurry. Place the vinyl template over the electrodes and drop cast 300 μL of 5.0×HA MIEC slurry onto each electrode. Wait until the MIEC slurry is fully cured to form a layer of the MIEC material 42 then repeat this step 6 times, so there are 6 total layers. Add more layers to fully cover the surface of the conductive thread and the copper mesh. Velcro straps were then sewn on the side of the fabric 30 so that it could be securely fasted as the armband 10 to a person's forearm.

The backside of the electrodes 20 on the Flex Neuroband can be coated with polyurethane (or other insulator) 44 as diagrammatically shown in the exploded view of FIG. 4 to eliminate any potential biting there might be from it if someone were to touch the backside during stimulations.

Able Body Feasibility Testing was conducted to determine if the thusly constructed Flex NeuroBand would be effective in evoking movements by functional electrical stimulation (FES). The setup was as follows: the exposed ends of the conductive threads were electrically connected to corresponding individual stainless steel electrodes. The arm was sprayed with Signa™ spray prior to testing. The FES stimulation tests were able to evoke 4 different movements with minimal biting. When subject 1 wore it, he got almost no biting, but when subjects 2 and 3 tried the device, they got a few sharp spots. This is likely due to the fact there was not ample contact on their arms as their arms are smaller than subject 1's arm. This could presumably be remedied by using an armband more closely fitted to the diameter of the individual subject's arm. The FES tests were able to evoke wrist flexion, ring flexion, middle/ring finger flexion, and hand open movements.

The Flex NeuroBand was lightweight, with its total weight, including all the wires, at around 18 grams. The Flex NeuroBand is easy to clean with a few isopropanol wipes. The Flex NeuroBand can be folded into a compact area as well. Notably, the flexible electrodes 20 were themselves highly flexible—a single electrode was capable of a full 180° fold back upon itself.

In addition to the FES tests just described, EMG measurement tests were also performed using the Flex NeuroBand.

To test the bio-signal recording capabilities of the Flex NeuroBand, it was configured to enable bipolar voltage recordings between two electrodes tied to an additional reference electrode on the Flex NeuroBand. Bipolar electromyography (EMG) signals recorded from the right forearm flexor muscles during rest and hand flexion were sampled at 3 KHz using an Intan Recording Controller (Intan Technologies, Los Angeles, CA). An electrode connected to the left-hand thumb was used as a reference for the bipolar amplifier. In these tests, rather than being wrapped around the arm, the forearm was pressed on the open-faced Flex NeuroBand to insure a strong contact between the skin and electrodes. Signa™ spray was also applied to the forearm before electrode contact. The electrodes of the Flex NeuroBand were connected to a custom-built EMG signal acquisition module, which in turn was connected to a laptop computer.

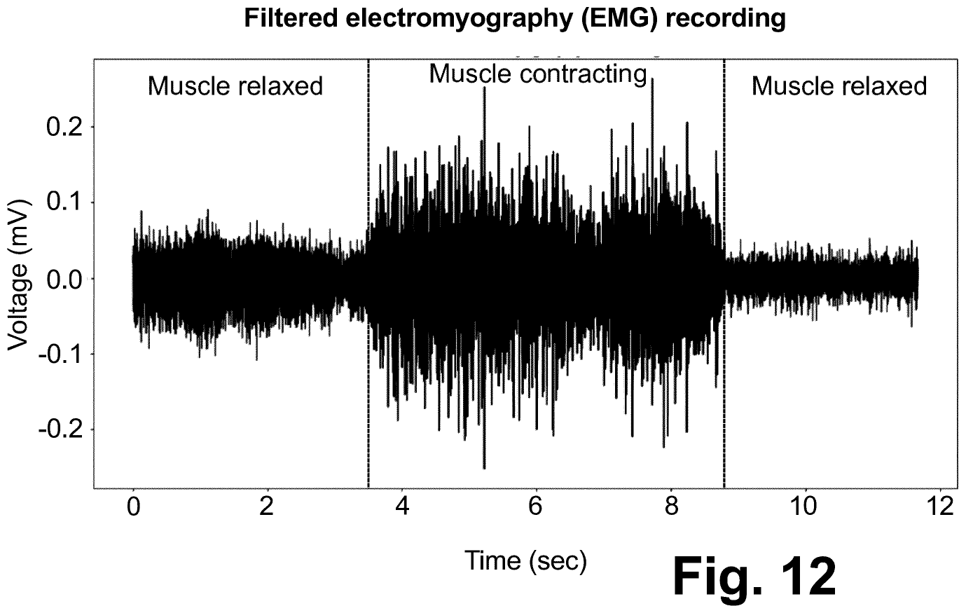

With reference to FIG. 12, EMG signal was recorded while a participant was cued to rest, then to contract flexor muscles, and then to rest again, all within about a 12 second duration as shown in the experimental plot of FIG. 12. The EMG data was bandpass filtered between 120-400 Hz using a 10th order Butterworth filter, and a 60 Hz notch filter was applied.

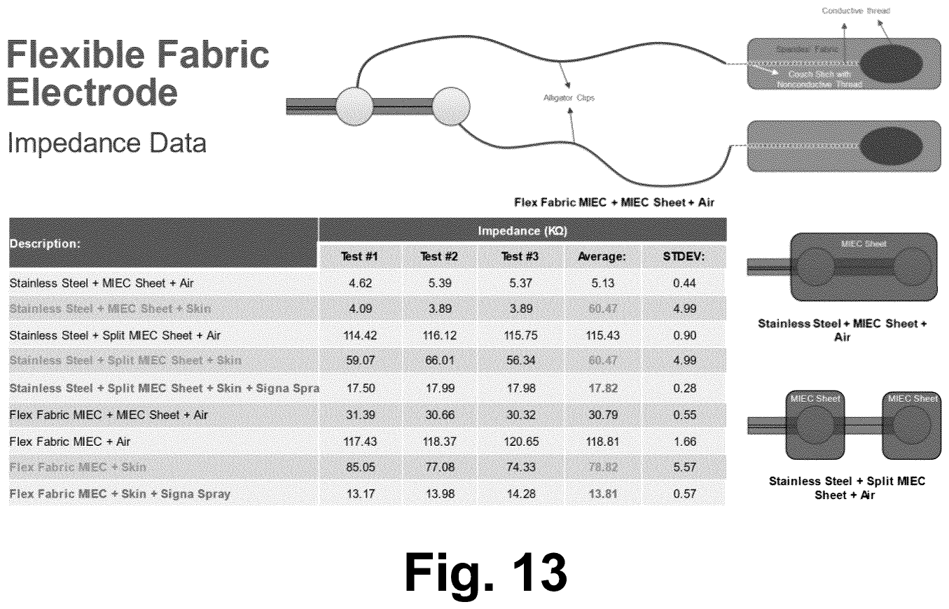

With reference to FIG. 13, impedance data of different setups was also tested and recorded, and these results are presented in FIG. 13. This data demonstrates that the flexible electrode 20 that includes the electrically conductive mesh 40 and MIEC material 42 on the skin and with Signa spray performed better (lower impedance) than a stainless-steel electrode with signa spray.

In the following, some further nonlimiting illustrative embodiments are described.

An illustrative embodiment is directed to a conductive garment, comprising: an elastic textile; a conductive mesh embedded into the textile; a conductive thread embedded in the textile and connected to the conductive mesh; and a conductive, elastic polymer electrode material contacting the conductive mesh.

An illustrative embodiment is directed to a conductive garment, comprising: an elastic textile; a plurality of flexible, elastic electrodes embedded into the textile; and conductive threads sewn into the elastic textile and connected to the plurality of flexible, elastic electrodes; wherein the flexible electrodes are conformable to a surface under the force of gravity and elastically bendable to 180°.

An illustrative embodiment is directed to a conductive electrode sleeve system, comprising: an elastic compression sleeve comprising an elastic fabric that is impregnated with a conductive polymer; a flexible electrode sleeve comprising a plurality of electrodes embedded in a flexible matrix; wherein the flexible electrode sleeve contacts and overlies the elastic compression sleeve.

An illustrative embodiment is directed to a method of forming the garments by 3D knitting. Mesh in apparel is elastic or deformable—able to be bent to a taco shape with sides bent 90° and, in some embodiments released to return to previous shape—deformable by gravity (one g). In some embodiments, the mesh is copper. In some embodiments the copper mesh is circular with a diameter in the range of 2 mm to 2 cm, 2 mm to 1.5 cm, 5 mm to 1 cm. In some embodiments, the copper mesh is electrically connected to a wire (such as a copper wire) that, in turn, can be connected to a controller (or connectable to an electrical potential). In some embodiments, the copper mesh is a thin screen and can be any mesh such as, but not limited to 10, 20, 30, 50, 70, 90 or 100 mesh. The wire diameter in the mesh may be, but is not limited to 1 mm or less, or in the range of 0.05 to 1 mm or 0.1 to 1 mm. In some embodiments, the wire is sewn into the copper mesh. The wire may for example comprise an electrically conductive core (e.g. copper) and a dielectric coating, but is uncoated in the region where the wire connects to the copper mesh.

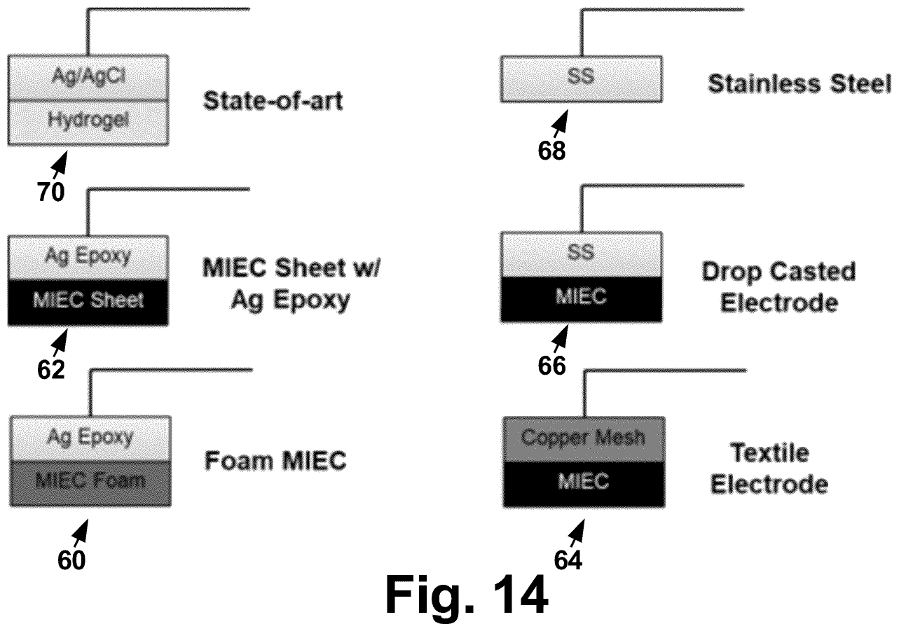

With reference to FIGS. 14-24, in the following some further experimental results are presented. The aim of these experiments was to uncover the capacity for altering the interfacial charge transfer and SNR of MIEC electrodes through the manipulation of electrode composition and form factors of the electrode. Four different form factors of dry MIEC electrodes were fabricated, as shown in FIG. 14, which included a MIEC foam electrode 60, MIEC elastomeric sheet connected with a flexible silver epoxy backing, electrode 62, MIEC fabric/textile electrode 64 that has the potential to be integrated into any textile, and MIEC coated stainless steel (SS) electrode 66. The benefit of these conformal, soft, dry MIEC electrodes 60, 62, 64, 66 allow for the elimination of application of electronic gel to skin for a conduction enhancer, ability to fine tune the interfacial charge transfer properties of the material, low cost, decrease weight, quick preparation time, potential for breathability when integrated in garment, potential for washability, increased electrode stability, biocompatible, all without losing functionality. For reference, FIG. 14 also diagrammatically shows a stainless steel electrode 68 and a hydrogel-coated Ag/AgCl electrode 70. The electrodes were characterized through electrochemical impedance spectroscopy (EIS), to determine the interfacial charge transfer properties on synthetic skin, and artificial EMG testing to determine the electrodes SNR.

Electrode fabrication was conducted through drop casting different MIEC composition on a stainless-steel button (0.4 wt. % SWNTs/1.1 wt. % HA (MIEC Coated SS 1), and 1.2 wt. % SWNTs/1.1 wt. % HA (MIEC Coated SS 2), 0.4 wt. % SWNTs/2.2 wt. % HA (MIEC Coated SS 3), 0.8 wt. % SWNTs/2.2 wt. % HA (MIEC Coated SS 4), 1.2 wt. % SWNTs/2.2 wt. % HA (MIEC Coated SS 5)), casting and MIEC sheet and cutting it down an electrode size of 12 mm and attaching a wire to the back of it using a flexible silver epoxy, impregnated low density polyurethane foam with the MIEC slurry, textile/fabric MIEC electrode, stainless-steel control, and Natus Ag—AgCl wet electrode.

To prepare a set of synthetic skin plates and synthetic skin plates with wires embedded, a solution of 4.5% w/v agar and 0.97% w/v NaCl was prepared in DI water. This solution was then heated via a 20-minute sterilization time liquid autoclave cycle to dissolve the agar powder. After cooling slightly (approx. 80° C.), 20 or 40 mL of the solution was then aliquoted into 100 mm×15 mm petri dishes. Additionally, 18 mL of the solution was aliquoted into 100 mm×15 mm petri dishes and allowed to cool for several seconds. The black and red twisted wires were placed on top of the agar layer with the exposed ends in the center of the plate. An additional 22 mL of solution was added on top of these wires. The wired were taped into place on the side of the petri dish to ensure they did not move while cooling or during use. All plates were allowed to fully cool until reaching room temperature and then stored inverted and sealed with parafilm at 4° C. until use.

The electrodes were subjected to controlled signal-to-noise ratio through artificial EMG. To compare signal quality objectively and accurately with each set of electrodes, an electrical phantom setup was used, which included a conductive material with embedded wires used to broadcast ground-truth electrical signals. In this instance, the conductive material was a stainless-steel electrode. The simulated EMG data were created using physiologically relevant parameters for human muscle that were previously used to test EMG technology with a phantom device (see Schlink, Bryan R and Daniel P Ferris, A lower limb phantom for simulation and assessment of electromyography technology. IEEE Transactions on Neural Systems and Rehabilitation Engineering, 2019. 27(12): p. 2378-2385). A pair of wires were embedded within the conductive gelatin to act as an antenna for broadcasting the simulated EMG signals.

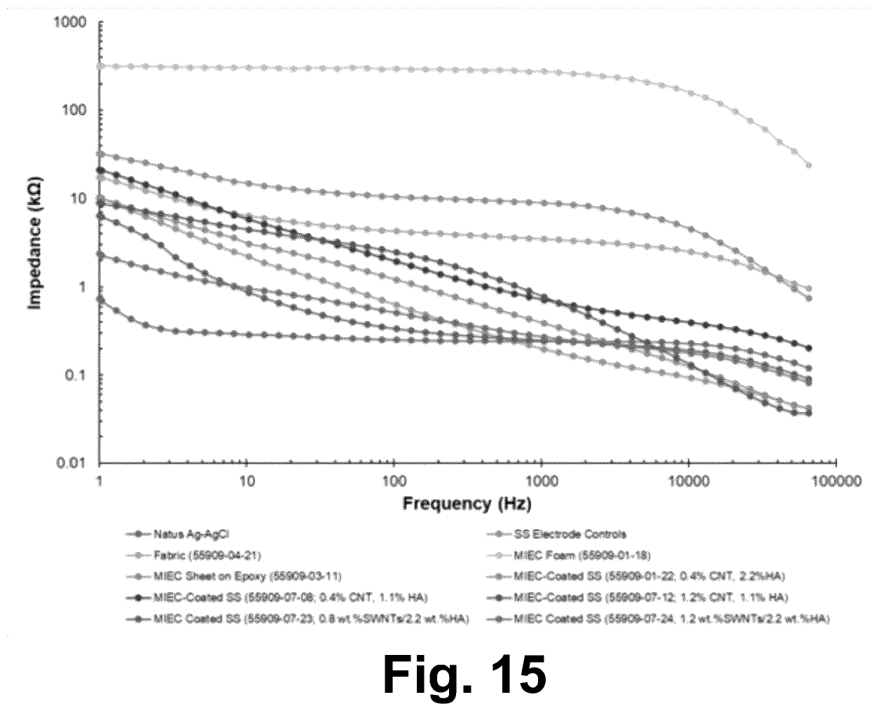

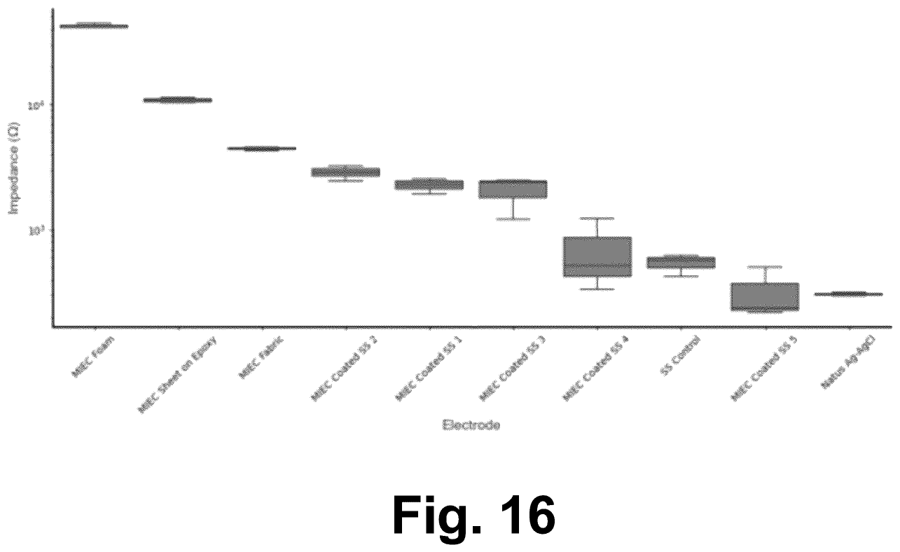

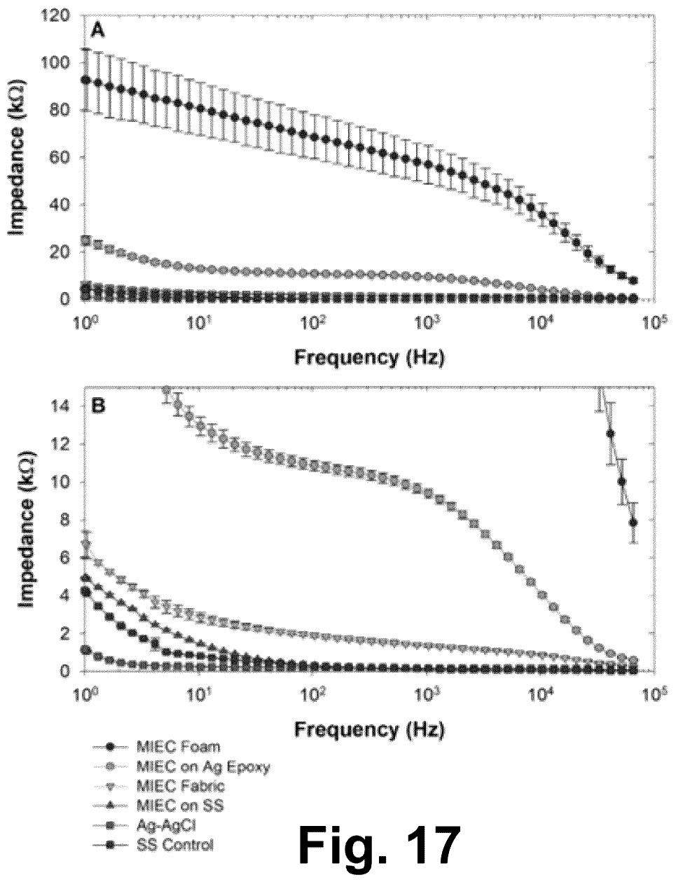

Impedance measurements were run with EIS. A 200-gram plate was placed on top of the electrodes, covered by a thin, rigid dielectric layer for shielding. The impedance vs. frequency sweep of these electrodes can be seen in FIG. 15 (Test 1). The corresponding Impedance at 100 Hz, which is the frequency for EMG, can be seen in FIG. 16 (Test 1). Impedance measurements were taken on various days with different synthetic skins used. The slight change in skin thickness and variation could result in slight error in the data. Impedance testing was then rerun with all the sample on the same synthetic skin imbedded with wires on the same day with environmental conditions at 20.1° C. and 40% relative humidity (Test 2). The corresponding impedance vs. frequency sweep can be seen in FIG. 17. Note, that the Test 2 did not include all MIEC on SS formulations as the other formulation had a worse SNR (which will be discussed later).

Differences in the setups between the two tests of impedance vs. frequency included initially physically pressing the electrodes down onto the synthetic skin in Test 2, which allowed the electrodes to make more intimate contact with the skin. The pressure was immediately taken off the electrodes and 200 g plates were placed on the electrodes for the duration of testing. During Test 1, there was no initial pressure from the electrodes to the skin, which may have affected the results as the electrodes may not have been making intimate contact with the skin.

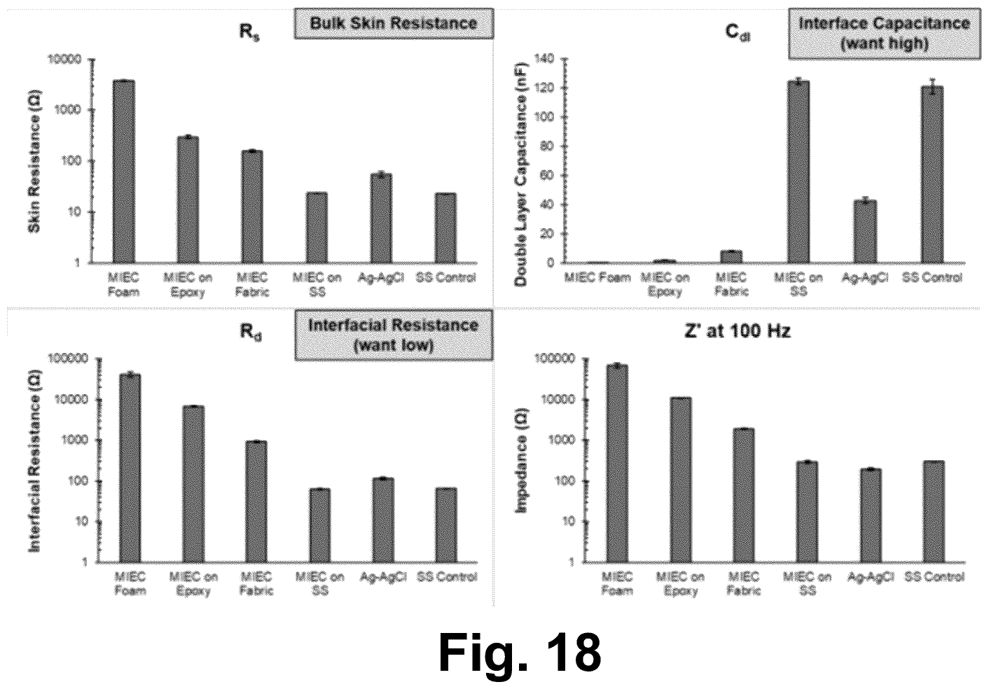

Interfacial Charge transfer was performed through EIS on the electrodes on the same surrogate skin used for artificial EMG testing. The Nyquist plot output was fitted by a Randles Cell model, which consists of a charge-transfer resistor (R_{d}) in parallel with a double layer capacitor (C_{dl}), and a solution or bulk resistance (Rₛ) in series. Interfacial charge transfer data of the electrodes on synthetic skin can be seen in FIG. 18. Lower Rₛ and R_{d} values, along with higher C_{dl} values, are expected to be preferred since they result in a lower total impedance (Z′) based on the least squares nonlinear fitting method that is used to estimate the values of the electrical circuit model and equation. See Lopes, Pedro Alhais, et al., Soft bioelectronic stickers: selection and evaluation of skin-interfacing electrodes. Advanced healthcare materials, 2019. 8(15): p. 1900234. EIS data predicts that the MIEC on SS electrode should have the best SNR, followed by Ag—AgCl, MIEC Fabric, MIEC on Epoxy, and MIEC on Foam.

It was noted that the foam MIEC electrode yielded the highest impedance. Foam electrodes can sometimes have air gaps or voids within the foam structure. These air gaps can act as insulators, preventing good electrical contact between the electrode and the skin. See Ng, Charn Loong and Mamun Bin Ibne Reaz, Characterization of textile-insulated capacitive biosensors. Sensors, 2017. 17(3): p. 574. Inadequate contact area between the electrode and the skin increases the impedance, resulting in reduced signal quality. See Yang, Liangtao, et al., Insight into the contact impedance between the electrode and the skin surface for electrophysical recordings. ACS omega, 2022. 7(16): p. 13906-13912. If the pressure of the foam electrodes to the synthetic skin was increased by adding more weight to the plastic dish when testing, the impedance would likely decrease. This approach is contemplated to be advantageous in EEG applications that involve wearing a helmet for monitoring of brain signals and activity. For EEG, the foam electrodes also have the advantage of not having the poor effect of wet hydrogel electrodes that can ruin one's hair and impede the electrodes contact area.

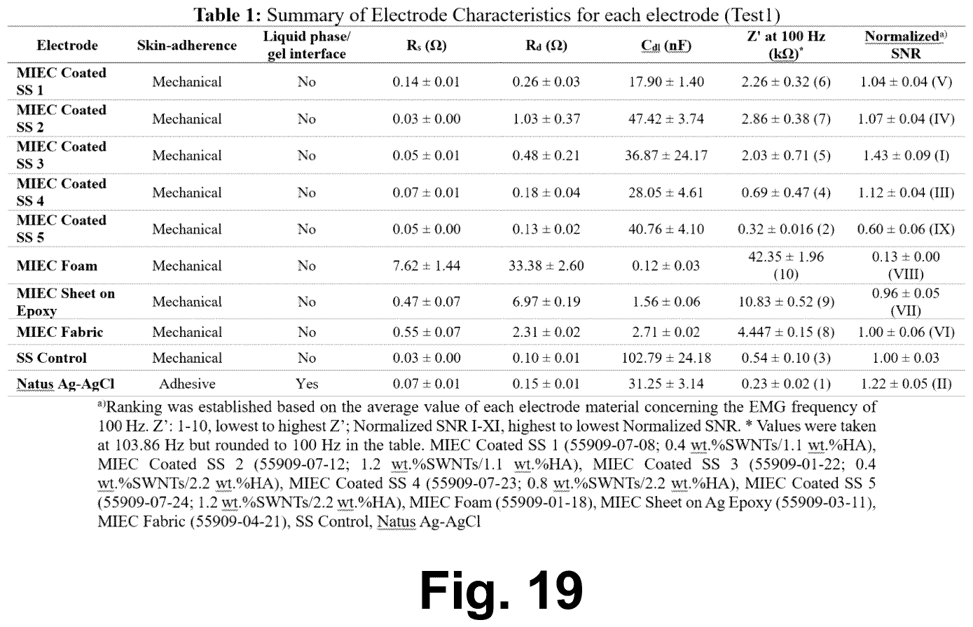

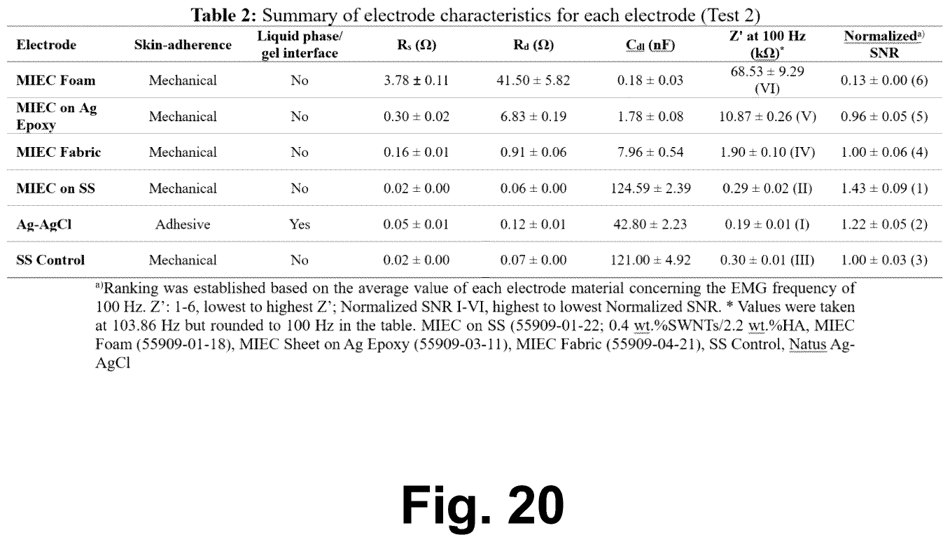

All tested electrodes had a low relative impedance during the frequency sweep, indicating that the tested dry MIEC electrodes in all form factors outperform current state of the art dry electrodes and work almost as well Ag—AgCl electrodes at certain frequencies. The fabric electrode and all MIEC coated on SS had an impedance lower than 5 kΩ, which is in the impedance range of 1 kΩ-10 kΩ for COTS wet electrodes and lower than 80 kΩ for COTS dry electrodes at 100 Hz. Cf. Yang, Liangtao, et al., Insight into the contact impedance between the electrode and the skin surface for electrophysical recordings. ACS omega, 2022. 7(16): p. 13906-1391; and Lopez-Gordo, Miguel Angel, Daniel Sanchez-Morillo, and F Pelayo Valle, Dry EEG electrodes. Sensors, 2014. 14(7): p. 12847-1287. The tested MIEC electrodes demonstrated impedance values lower than 10 kΩ at frequencies of 10 Hz, which is the frequency commonly used for EEG recordings. See Górecka, Joanna and Przemystaw Makiewicz, The dependence of electrode impedance on the number of performed EEG examinations. Sensors, 2019. 19(11): p. 2608. These results are comparable to state-of-the-art electrodes, demonstrating high-quality performance. Electrode interfacial charge transfer values, impedance at 100 Hz, and Normalized SNR (which will be discussed later herein) are shown in Table 1 (Test 1) presented in FIG. 19 and Table 2 (Test 2) presented in FIG. 20.

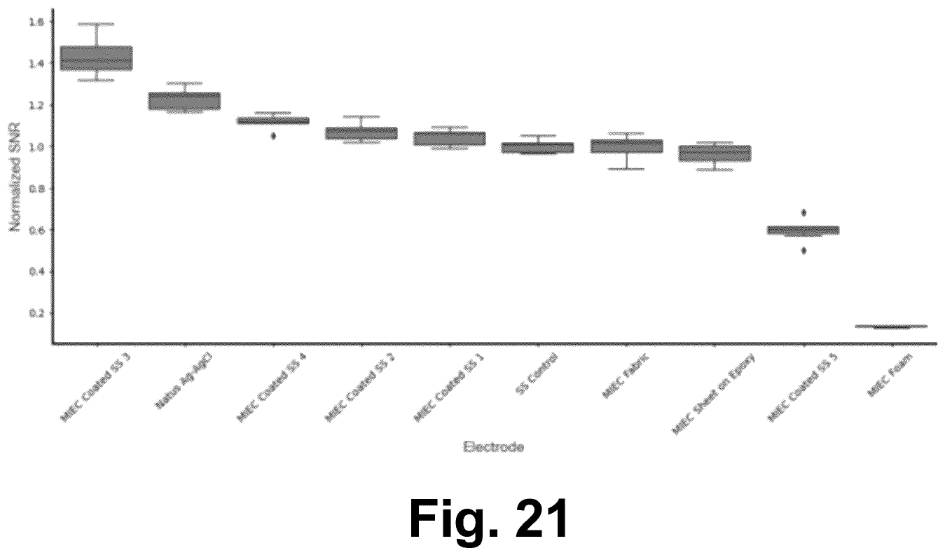

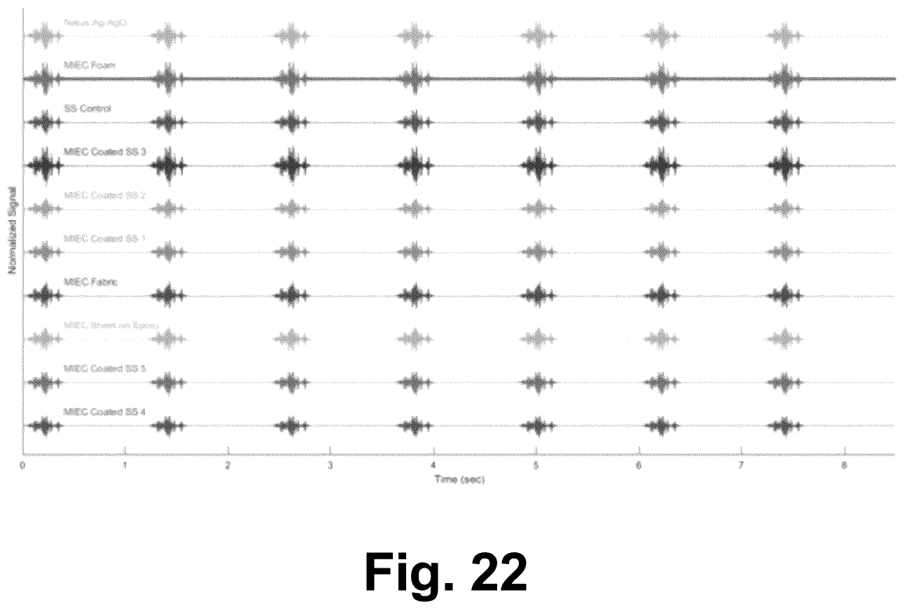

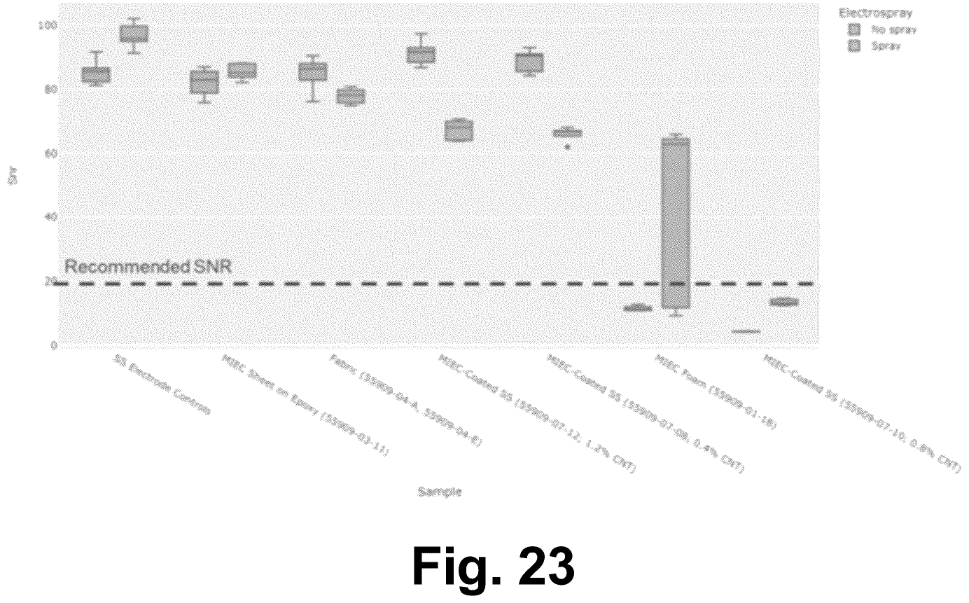

Normalized SNR was calculated for all electrodes using the artificial EMG setup. Some electrodes were run on different days between two synthetic skins (synthetic skins were fabricated with the same batch just poured into different containers when cured). All SNR was normalized to the stainless-steel reference electrode 68 (see FIG. 14) which served as the standard dry electrode for these tests. The Ag—AgCl electrode 70 (see FIG. 14) was unable to be used as the reference electrode due to the silver chloride leaching out into the synthetic skin when placed on the synthetic skin for prolonged time. Normalized SNR can be seen in FIG. 21 and the normalized signal plot can be seen in FIG. 22. All data can be seen in Table 1 (Test 1) and Table 2 (Test 2) (presented in FIGS. 19 and 20 respectively).

These tests demonstrate that the MIEC dry electrodes performed very well for SNR. Compared to the state-of-the-art electrodes 68, 70, the MIEC on SS electrode 66 had a higher normalized SNR.

The above-described testing was carried out without spraying the electrodes with conductive spray. With reference to FIG. 23, SNR of the electrodes with and without conductive spray is presented.

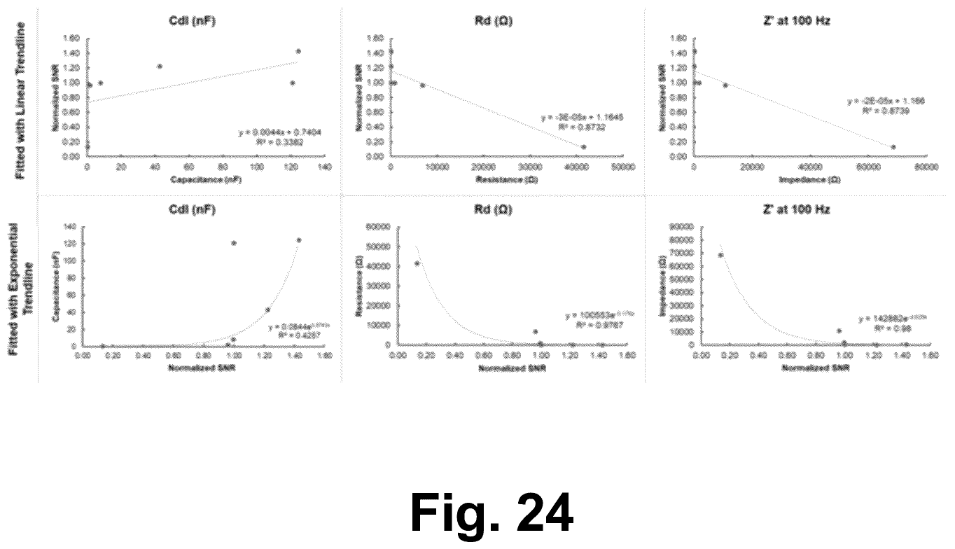

With reference to FIG. 24, trends between the interfacial charge transfer of the electrodes in respect to normalized SNR were investigated and the results are presented. The plots of FIG. 24 are presented with either linear or exponentially fitted trendlines to make a convenient analysis as to whether the normal SNR and interfacial charge transfer properties of the electrodes are correlated.

The properties of the electrodes and skin that had the most significant impact on the SNR of the tested dry MIEC electrodes were having a low bulk skin resistance, low electrode interfacial resistance, and low electrode-to-skin interfacial impedance. It is noted that the conductivity of the electrodes did not have a significant effect on the SNR of the electrodes. The interfacial capacitance of the electrodes was not influential on the materials SNR. The double layer capacitance could be fitted using a C_{dl} with a Helmholtz layer and the diffuse layers model to obtain a more accurately represented component that could lead to a more concrete correlation to the SNR value of the electrodes. Divergence in the interfacial charge transfer properties of the electrodes can potentially arise from factors such as variances in electrode positioning, fluctuations in electrode mass, inadequate pressure application onto the electrode, variations in synthetic skin temperature, relative humidity, slight discrepancies in skin thickness among samples, and inherent distinctions in batch properties between the acquired SWNTs and HA from the supplier. The results presented with reference to FIGS. 14-24 show a clear trend with high double layer capacitance, low interfacial charge resistance (polarization resistance), and low impedance result in the best SNR, which can be seen in FIG. 24.

In summary, in the tests presented herein with reference to FIGS. 14-24, the tested dry MIEC electrode material outperformed the state-of-the-art “wet” (Ag—AgCl) electrode 70 and the “dry” (Stainless-Steel) electrode 68 for SNR.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. An electronic garment comprising:
an elastic textile garment configured to be worn on anatomy of an associated wearer, the elastic textile garment comprising an elastic textile and having an inner surface arranged to contact the anatomy when the elastic textile garment is worn on the anatomy;
electrodes secured to the inner surface of the elastic textile garment, each electrode including an electrically exposed portion of an insulated electrically conductive thread sewn onto or into the elastic textile garment; and
an electrically conductive polymer electrode material arranged to contact the electrodes.

2. The electronic garment of claim 1 wherein each electrode further includes:
a flexible electrically conductive layer sewn onto the inner surface of the elastic textile garment, the electrically exposed portion of the insulated electrically conductive thread being electrically connected with the flexible electrically conductive layer.

3. The electronic garment of claim 2 wherein the flexible electrically conductive layers of the electrodes are electrically conductive meshes.

4. The electronic garment of claim 3 wherein the electrically conductive meshes comprise metal meshes or carbon nanotube fiber textile meshes.

5. The electronic garment of claim 2 wherein each flexible electrically conductive layer is capable of a full 180° fold back upon itself.

6. The electronic garment of claim 1 wherein the elastic textile garment is formed by three-dimensional (3D) knitting or weaving including incorporating the electrically conductive threads into the elastic textile garment during the 3D knitting or weaving.

7. The electronic garment of claim 1 wherein the electrically conductive thread of each electrode comprises:
an electrically conductive core; and
an electrically insulating coating that coats the electrically conductive core;
wherein the electrically exposed portion of the electrically conductive thread comprises a stripped end of the electrically conductive thread.

8. The electronic garment of claim 1 wherein the electrically conductive polymer electrode material comprises a mixed ionic-electronic conducting (MIEC) material.

9. The electronic garment of claim 8 wherein the MIEC material comprises individual MIEC material regions disposed on respective electrodes.

10. The electronic garment of claim 8 further comprising:
a compression sleeve liner coated and/or infused with the MIEC material, the compression sleeve liner configured to be worn on the anatomy underneath the elastic textile garment.

11. The electronic garment of claim 8 wherein the MIEC material comprises a mixture of an electrical conductor and an ionic conductor.

12. The electronic garment of claim 11 wherein the electrical conductor of the MIEC material comprises carbon nanotubes and the ionic conductor of the MIEC material comprises a glycosaminoglycan.

13. The electronic garment of claim 11 wherein the MIEC material comprises an interconnected network of the electrical conductor and the ionic conductor in an elastomeric matrix.

14. The electronic garment of claim 1 further comprising a scent added to the elastic textile garment.

15. An electrical stimulation system comprising:
an electronic garment as set forth in claim 1; and
an electrical simulator connected with the electrodes of the electronic garment via the electrically conductive threads of the electronic garment, the electrical stimulator configured to apply transcutaneous electrical nerve stimulation (TENS) or neuromuscular electrical stimulation (NMES) via the electronic garment to the anatomy on which the electronic garment is worn.

16. An electrophysiology readout system comprising:
an electronic garment as set forth in claim 1; and
electrophysiology signal readout electronics including an electrophysiology signal amplifier connected with the electrodes of the electronic garment via the electrically conductive threads of the electronic garment, the electrophysiology signal readout electronics configured to read electrophysiology signals from the anatomy on which the electronic garment is worn via the electronic garment.

17. A method of manufacturing an electronic garment, the method comprising:
providing an elastic textile garment configured to be worn on anatomy of an associated wearer, the elastic textile garment comprising an elastic textile and having an inner surface arranged to contact the anatomy when the elastic textile garment is worn on the anatomy;
sewing electrodes comprising electrically exposed portions of electrically conductive threads onto the elastic textile garment; and
arranging an electrically conductive polymer material to contact the electrodes.

18. The method of claim 17 wherein the electrically conductive polymer electrode material comprises a mixed ionic-electronic conducting (MIEC) material.

19. The method of claim 17 wherein the electrodes further include electrically conductive meshes, and the sewing of the electrodes onto or into the elastic textile garment includes:
sewing the electrically conductive threads onto or into the elastic textile garment; and
sewing the electrically conductive meshes onto the elastic textile garment; and
wherein the exposed portions of the electrically conductive threads are electrically connected with the electrically conductive meshes.

20. The method of claim 17 wherein the providing of the elastic textile garment and the sewing of the electrodes onto the elastic textile garment are performed concurrently by three-dimensional (3D) knitting.
